(19) United States
(12) Patent Application Publication
(10) Pub. No.: US 2015/0209533 A1
(43) Pub. Date: Jul. 30, 2015

(54) RESPIRATORY ASSISTANCE DEVICE, NASAL APPLIANCE AND RESPIRATORY ASSISTANCE MASK

(71) Applicant: Georges BOUSSIGNAC, Antony (FR)

(72) Inventor: Georges Boussignac, Antony (FR)

(21) Appl. No.: 14/424,758

(22) PCT Filed: Aug. 28, 2013
(86) PCT No.: PCT/FR2013/051979

§ 371 Date: Feb. 27, 2015

(30) Foreign Application Priority Data
Aug. 28, 2012 (FR) 1258034

(51) Int. Cl. A61M 16/00; A61M 16/06; A61M 16/14; A61M 11/02

(52) U.S. Cl. CPC A61M 16/0096; A61M 11/02; A61M 16/0069; A61M 16/0666; A61M 16/14; A61M 2202/04; A61M 2205/3334

(57) ABSTRACT

A device for respiratory assistance of a patient includes a tubular element forming a main channel for connection by its distal end to a respiratory tract of the patient, the main channel connecting the patient's respiratory system by its proximal end to the outside, the device further including at least one auxiliary channel for injecting a jet or jets of respirable gas through one or more distal outlet holes of the auxiliary channel or channels, the one or more outlet holes opening-out in the main channel in the vicinity of the distal end of the latter, deflecting elements for deflecting the jets of respirable gas toward the interior of the main channel. The diameter of the outlet hole of each auxiliary channel is less than 150 microns. The outlet holes of the device are shaped as an annular or semi annular strip resulting from assembling/uniting together certain distal outlet holes.

### FIELD OF THE INVENTION

The present invention relates to an improved device for respiratory assistance to be used on patients whose spontaneous respiration is absent or insufficient, whether or not they are placed under artificial respiration. It is particularly well adapted to the devices for respiratory assistance invented by Mister Georges BOUSSIGNAC.

### BACKGROUND OF THE INVENTION

The principle that the device for respiratory assistance invented by Mister BOUSSIGNAC is based on consists in injecting at least one pressurized respirable gas via a plurality of auxiliary channels into the lumen of a tube connected to the respiratory tract of the patient. That device for assistance is particular in being of the so-called open type, which means that the tube can remain open to the ambient air allowing thus for passing probes or other accessories, called herein after the ancillaries, for survey or treatment of the patient. More precisely, the device for assistance is formed from a tubular element or tube forming a main channel and which is intended to be connected by its distal end to a respiratory tract of a patient so that the main channel connects the patient's respiratory system to the outside. The device further comprises at least one auxiliary channel allowing for injection of a jet of respirable gas intended for the ventilation of said patient and leading to/opening-out by a distal outlet hole in that main channel in the vicinity (i.e. next to or close to) of the distal end of the latter. In that device for assistance, in front of the distal outlet hole of each auxiliary channel, means for deflecting, i.e. deflectors, said jets of respirable gas or gases toward the interior of the main channel are provided.

Thus, the jet of pressurized respirable gas passing through the auxiliary channel is deflected toward the axis of the main channel when it enters it. Downstream of said deflection means, i.e. inside the main channel, the pressure of said jet of respirable gas decreases and the jet gets out through the distal end of the tubular element at a low pressure. Experience has shown that downstream the distal outlet, pressure is low and maintained constant throughout the whole respiratory space. Further, that pressure depends from the flow rate of respirable gas in the auxiliary channels, and thus, there is a simple way of varying the pressure by acting on the flow rate of the gas in the auxiliary channels.

That device for assistance for which further information can be obtained from the French Patent Application FR-89/04280, has been improved several times, as can be seen for example in the following documents (list not complete):

EP0390684, WO2008113913, EP2228088, FR2942967, WO2007118973, FR2911073, FR2813197, WO03039638, FR2827778, WO0010632, EP0978291, and EP0701834.

It will be seen that those improvements can be implemented in the present invention. Especially, nebulization of liquid products, assistance for reanimation, nasal use, double flow, restriction of the inhalation flow in case of massage of the thoracic cage of a patient suffering from cardiac and respiratory arrest . . . .

That device has given a real comfort to the patients and to the medical personnel since it is of the open type. Further, it is relatively simple as to its working principle and simple to be produced.

However, the device such as it has been proposed has the inconvenience of generating a certain noise due to the injection of air which creates turbulences inside the device and since the latter is open.

A simple solution would be to add an acoustic filter or silencer on the opening of the distal end of the device, said filter or silencer allowing or not for passing the ancillaries, and in the latter case it should be removable, in order to be removed for passing the ancillaries during interventions. That simple solution implies however the inconvenient of making that device more complex or of adding an accessory to the device.

Applicant has remarked that, surprisingly, the fact of reducing the diameter of the outlet holes of the jets and/or that of the auxiliary channels for respirable gas and, at the same time, of increasing the number of jets or, in an equivalent manner, to unit together the outlet holes of the jets as an annular strip or as semi annular strips of reduced thickness (=width), each strip forming then an elongate outlet hole of its own, allows to reduce significantly the generated noise.

More precisely, le fact of passing from a diameter of the outlet holes of the auxiliary channels in the order of 400 to 800 microns (=micrometers) as described in the patent application FR89/04280, to a diameter of less than 150 microns and, even better, to a value comprised between 100 and 5 microns, allows to substantially reduce the generated noise. In the case of the strips, the thickness of each strip corresponding to an outlet hole of its own is then less than 150 microns and, even better, comprised between 100 and 5 microns.

That reduction of the dimensions of the outlet hole or holes of the jets, whether they are singular (having a reduced diameter) or shaped as strips (having a reduced thickness) has the supplemental advantage, besides the reduction of noise, to reduce the consumption of respirable gas, generally oxygen, of the jets produced by the outlet holes. Thus, it was possible to reduce the consumption of oxygen by 50% in a device having outlet holes with reduced diameter with acceptable results compared to those obtained with traditional devices with usual diameters of the outlet holes of the jets and a usual consumption of oxygen. That phenomenon can be explained by the modification of the speed of the jets and of the flow conditions of the gas resulting from the smaller outlet holes.

Contrarily to what one could have been afraid of, that reduction of the diameter does not imply any risk of clogging/obstruction of the outlet holes or of the auxiliary channels, thanks to the high quality of the gases now available. It also is possible to install a particulate filter at the arrival of the respirable gas in case of fear or doubt concerning the cleanliness of the gas.

It has to be stated that the documents of prior art, especially EP-2,228,088, FR-2,942,967, EP-2,239,004, and FR-2,980,978, mention auxiliary channels for forming jets, channels which it is said about that they have a diameter “in the order of 5 to 800 microns”. Independently from the fact that those values relate to the auxiliary channels and not to the outlet holes of the jets, the lower value “5” is practically incompatible with the rest of the descriptions of those documents, and one should normally read “500 to 800 microns”. Indeed, the value “5” of “5 to 800 microns” in those documents does not allow for producing a practically usable device, since between three and nine auxiliary channels only are foreseen (cf. [0042] of document EP-2,228,088) with a pressure of 0.5 bars ([0048] of document EP-2,228,088) which does not allow to obtain the indicated flow rate of 0.5 l/min ([0048] of document EP-2,228,088).

### SUMMARY OF THE INVENTION

The invention thus relates to a device for respiratory assistance of a patient comprising a tubular element forming a main channel intended for being connected by its distal end to a respiratory tract of the patient, said main channel connecting the patient's respiratory system by its proximal end to the outside, said device further comprising at least one auxiliary channel allowing for injection of a jet or of jets of respirable gas through one or more distal outlet holes of the auxiliary channel or channels, said jet or jets of respirable gas being intended for ventilation of the patient, said one or more outlet hole opening in the main channel in the vicinity of the distal end of the latter, deflecting means, i.e. deflectors, allowing to deflect the jets of respirable gas toward the interior of said main channel.

In that device, the diameter of at least the outlet hole of each auxiliary channel is less than 150 microns.

In the case of outlet holes arranged as a strip, the device comprises at least one outlet hole shaped as an annular strip or as semi annular strips resulting from the assembly/uniting together at least certain ones of the distal outlet holes, the thickness of the outlet holes shaped as a strip being less than 150 microns.

It shall be noted that one mentions assembling/uniting together the outlet holes, since in the means for producing jets of respirable gas, at least the distal outlet holes of jets are joint/united together as one or more annular strips or semi annular strips, and according to variants, the auxiliary channels also are joint/united together forming thus auxiliary channels shaped as one or more annular or semi annular strips.

More precisely, the device comprises at least one outlet hole shaped as an annular or semi annular strip resulting from uniting together at least certain ones of the distal outlet holes, the thickness of the strip-shaped outlet hole being less than 150 microns.

According to different implementations according to the invention, the following means, which can be used individually or according to any technically possible combination, are used:

the outlet holes are individual punctual holes,

the diameter of each outlet hole is less than or equal to 150 microns,

the diameter of each outlet hole is less than or equal to 100 microns,
the diameter of each outlet hole is comprised between 50 microns and 10 microns and is preferably about 25 microns,

the diameter of each outlet hole is comprised between 20 microns and 5 microns and is preferably about 10 microns,

the diameter of each outlet hole is comprised between 150 microns and 10 microns,

the diameter of each outlet hole is comprised between 150 microns and 100 microns,

the auxiliary channels have at least on a part of their way a diameter which is approximately equal to the one of their outlet holes,

a given auxiliary channel ends at a single outlet hole,

a given auxiliary channel ends at several outlet holes,

the outlet holes are located at the distal ends of the auxiliary channels,

the outlet holes are offset with respect to the distal ends of the auxiliary channels, said distal ends of the auxiliary channels beyond the outlet holes being dead-ends,

the diameters of the outlet hole and of the corresponding auxiliary channel are identical,

at least the distal part or distal parts of the auxiliary channel or auxiliary channels leading to/opening-out in the main channel is or are parallel to the latter,

the device comprises a plurality of auxiliary channels, said auxiliary channels being approximately parallel to the central axis of said tubular element on at least a part of their way,

the auxiliary channel or auxiliary channels are arranged in the wall of the tubular element,

the device comprises a number of auxiliary channels, each auxiliary channel forming a tubular passage way in the wall of the tubular element,

the device comprises a single common auxiliary channel to which the outlet holes are connected, said common auxiliary channel being an annular passage way or a distribution ring which is external, approximately coaxial and parallel to the central axis of the tubular element,

at least certain ones of the outlet holes are united together such as to form a single annular-shaped outlet hole or a segment of a ring the opening width/thickness of which corresponding to the diameter of an individual punctual hole, i.e. a width/thickness of less than 150 microns,

in the case of a single auxiliary channel forming an annular passage way that is coaxial to the main channel, the outlet holes are united together so as to form a single annular outlet opening-out in the main channel, the annular outlet having a width/thickness corresponding to the diameter of an individual punctual hole,

the outlet holes are chosen between the individualized punctual holes and/or the outlet holes united together as a strip-like, preferably annular or semi annular opening,

certain outlet holes which are adjacent to one another are united together such as to form a single outlet shaped as a part of a ring (=semi annular) opening-out in the main channel, the outlet shaped as a segment of a ring having a width/thickness corresponding to the diameter of an individual punctual hole,

the device comprises an outlet hole shaped as an annular strip,

the device comprises several outlet holes shaped as semi annular strips,

the thickness of each outlet hole shaped as an annular or semi annular strip is comprised between 50 microns and 10 microns and is preferably about 25 microns,

the outlet hole or holes shaped as a strip result from uniting together at least certain ones of the distal outlet holes and of their corresponding auxiliary channels,

the device comprises in addition to one or more outlet holes shaped as semi annular strips individualized punctual outlet holes,

the punctual holes have a diameter of less than 150 microns,

the diameter of each individualized punctual hole is comprised between 50 microns and 10 microns and is preferably about 25 microns,

in the case of outlet holes united together, the corresponding auxiliary channels are united together, too, forming an annular or semi annular auxiliary channel on their entire length or on part of it,

the outlet hole is formed in a first face diverging from the main channel and the deflection means is formed by an inclined second face of said main channel, located in front of the first face and converging toward the outlet hole,

the terminal portion of the main channel on the side of the distal end is widened,

the second face is extended toward the distal end of the main channel by a wall slightly broadening the main channel,

the deflection means is formed directly in the internal wall of the tubular element,

the deflection means is formed on a flange added to the distal end of the tubular element,

the deflection means consists of a discontinuous number of recesses of generally conical shape which are arranged in the internal wall, and at the bottom of each of them the distal end of an auxiliary channel ends by its outlet hole,

the device comprises a plurality of auxiliary channels, some of which are supplied commonly with a pressurized respirable gas,

the auxiliary channels which are not commonly supplied serve for introducing additional gaseous products such as medicine products or humid gases,

at least parts of the auxiliary channels are commonly supplied with respirable gas by means of a distribution ring which is coaxial to the tubular element,

at least another part of the auxiliary channels are commonly supplied with medicine products or humidity,

the terminal portion of the main channel on the side of the distal end comprises a restricted portion,

the terminal portion of the main channel on the side of the distal end comprises a restricting ring,

the outlet holes of the auxiliary channels are arranged as a crown,

the device comprises at last one crown of outlet holes of auxiliary channels, the outlet holes of a crown being located along a transverse section of the tubular element,

the device comprises at least two crown of outlet holes of auxiliary channels which are offset along said tubular element, the outlet holes of different crowns coming from identical auxiliary channels, and the same auxiliary channel may output in several outlet holes,

the device comprises at least two crowns of outlet holes of auxiliary channels which are offset along said tubular element, the outlet holes of different crowns coming from different auxiliary channels, and a given auxiliary channel outputs in a single outlet hole,

the outlet holes of a crown or part of them are united together to form a common annular or semi annular hole,

the device comprises at least two crowns of outlet holes shaped as annular or semi annular strips,

the device having crowns of holes comprises, in addition to its outlet hole or holes shaped as annular or semi annular strips, punctual outlet holes,

the deflection means allows the jets of respirable gas of the auxiliary channels to converge to one another inside the main channel,

the deflection means are arranged as to allow for orienting the jets toward the distal end of the tubular element with an inclination with respect to the central axis of said tubular element which is between 90°, the axis of the jets then being perpendicular to said central axis, and 25°, the axis of the jets then crossing the central axis with an angle of 25°,

all jets have the same inclination,

the jets have different inclinations according to the outlet holes,

the jets have different inclinations according to the crown of outlet holes,

the inclination of the jets is about 45°,
and/or for certain variants:

the device is arranged as a two-inverted-flow device, allowing for favoring both exhaling and inhaling of a patient, the device comprising at least one supplemental auxiliary channel, which is independent from the first auxiliary channel or channels of the jets of the distal end of the main channel and connected to a source of pressurized gas, said at least one supplemental channel leading to/opening-out in the main channel in the vicinity of the proximal end of the latter, whereas in front of each proximal outlet hole of the corresponding supplemental auxiliary channel, means, i.e. deflectors, are provided for deflecting the gas jet passing through the latter toward the interior of said main channel,

at least the end or ends of the supplemental auxiliary channel or channels, which lead to/open-out by respective outlet holes in the main channel, is or are parallel to the latter,

preferably, in the case of a two-inverted-flow device with supplemental auxiliary channel, the proximal part of the device is roughly the same or even identical, symmetrically, to the distal part of that device, at least as to where said auxiliary channels and said deflection means are located,

the device constitutes the inlet and outlet flange for air of a mask for respiratory assistance intended to be applied the face of a patient,

the device constituting the inlet and outlet flange of a mask for respiratory assistance is removable,
and/or for certain variants:

the device further comprises deriving means which are capable of deriving a part of the volume of said respirable gas intended for said auxiliary channel before entering into the latter and means for sucking in ambient air by said derived portion of respirable gas, and said sucking means are connected to the main channel so that the sucking means are able to guide, in that main channel, the ambient air sucked in and mixed with said derived portion of respirable gas,

the device comprises means for adjusting the portion of respirable gas derived by said means for deriving,

the means for adjusting the portion of derived respirable gas are located between said means for deriving and the means for sucking in ambient air,

the means for adjusting the portion of derived respirable gas comprises at least one valve,

the device further comprises means for adjusting the flow rate of diluted respirable gas coming out of said sucking means and intended to enter the main channel,

the means for adjusting the flow rate of diluted respiratory gas are arranged between said sucking means ambient air and means for fluid communication,

the means for adjusting the flow rate of diluted respirable gas comprises at least one valve,

the flow of diluted respirable gas coming out of said sucking means passes into the main channel through a communication hole which is arranged in the wall of the device,

the main channel is formed by a tube,

the main channel is formed by a flexible tube,

the flow of diluted respirable gas coming out of said sucking means passes through a tight flexible hose which surrounds, at least on a part of its length, said flexible tube forming the main channel and which forms a peripheral way around said flexible tube and in which ends the communication hole,

the sucking means are directly mounted on said flexible tube in the vicinity of its proximal end,

the communication hole is located between the deflection means of the jets and the distal end of the main channel,

the communication hole is divided into a number of communication holes,
and/or, for certain variants:

the device further comprises means for spontaneously slowing down the intake of ambient air into the main channel via its proximal end,

the means for slowing down the intake of ambient air into the main channel comprises a hollow body provided with a first valve and a second valve which are normally closed, the first valve being adapted for opening spontaneously and immediately in case of an overpressure or counterpressure on the side of the respiratory tract of the patient, especially during a thoracic compression, whereas the second valve is adapted for spontaneous, but progressive opening other than in case of said overpressure or counterpressure, especially in case of suppression of a thoracic compression, and the hollow body is located at the proximal end of the tubular element of the device,

the first and second valves are located parallel to one another between the outside and the internal cavity of the hollow body,

the first and second valves are located in series between the outside and the internal cavity of the hollow body, one of these valves being held by the other one,

the first valve is formed by an elastic membrane which presses spontaneously against a valve seat provided in said hollow body and which is attached to the seat by fixing points located around its periphery, the air pushed out during overpressure or counterpressure passing in a free manner from the cavity of the hollow body to the outside through passage ways which are formed spontaneously and immediately by the elastic deformation of said membrane between said fixing points and said seat, and the second valve is formed by at least one slot having joined edges formed in said membrane, the air inhaled during suppression of the overpressure or counterpressure passing progressively, by being slowed down, from the outside to the cavity of the hollow body through the passage way that is formed spontaneously in said membrane by elastic deformation of the latter under progressive spacing of the joined edges,

the means for slowing down the intake of ambient air into the tubular element of the device is an integral part thereof,

the means for slowing down the intake of ambient air into the tubular element of the device is mounted to it in a removable manner,
and/or for certain variants:

the tubular element forming the main channel of the device comprises at least a lateral security hole which goes through its side wall at least approximately to the converging point of the jets of respirable gas or gases and which is arranged to connect to the outside that part of said main channel which is located at the distal and downstream side with respect to the direction of the jets of respirable gas or gases and with respect to the deflection means,

the distal part of the main channel communicates directly with the outside through said lateral security hole,

the lateral security hole is closed by a removable stopper,

the lateral security hole is closed by a removable captive stopper,

the device comprises fibrous or porous means for masking the noise of the jets of respirable gas passing through said lateral security hole,

the device comprises a conduit connecting the lateral security hole to the outside,

the fibrous or porous means is inside the conduit,

the conduit is constituted of a coaxial conduit surrounding the tubular element,

the coaxial conduit ends on the outside at the side of the proximal end of the tubular element,

the coaxial conduit ends on the outside at the side of the distal end of the tubular element,

the conduit is constituted of a flexible hose surrounding the tubular element and ending on the outside at the side of the proximal end of said tubular element,

the conduit is constituted of a flexible hose surrounding the tubular element and ending on the outside at the side of the distal end of said tubular element,
and/or for certain variants:

in order for the device to be able to deliver to the respiratory tract of the patient a predetermined flow rate of respiratory gas or gases under a pressure for use having a value that has to be comprised in a range of values for use which make sure that the device works efficiently without any danger for the patient, said device being connected to the source of respiratory gas or gases by a feeding conduit for gas and at a feeding pressure comprised between a minimum value and a maximum value, the feeding conduit for respiratory gas or gases comprises a specific element for load loss making sure that said pressure for use is at most equal to the upper value of the range of values for use when the feeding pressure is at the maximum value,

the specific element for load loss is constituted of a stopper which is arranged for closing the feeding conduit for respiratory gas and which is pierced to provide a longitudinal passage way for the gas or gases,

the value of the load loss introduced by said specific element for load loss is adjusted by the length of the pierced stopper,

the specific element for load loss is constituted of a portion of a profile,
and/or for certain variants:

the device comprises, between the deflection means and the distal end of the main channel, a communication means which is controllable as to opening and closing and is arranged as to form, when it is in the open position, a passage way connecting the main channel to the external environment,

the passage way connecting the main channel to the external environment has a variable cross section,

the communication means is of the type of a laterally pierced turning ring which is arranged for uncovering passage ways of different diameters,

the turning ring is mounted directly on the tubular element,

the turning ring is mounted on a so called chimney communicating with the main channel,
and/or for certain variants:

the device further comprises at least one feeding conduit supplied with a liquid product to be nebulized in the main channel and means for nebulization,

the device further comprises at least one added feeding conduit supplied with a liquid product to be nebulized and lodged in the main channel, said feeding conduit penetrating into the main channel through the proximal end of the tubular element and ending in the main channel in the vicinity of where, on the latter, the outlet holes of the auxiliary channel or auxiliary channels open-out,

the internal diameter of the added feeding conduit is of the order of 200 to 300 microns,

at the outside of the tubular element, the added feeding conduit is ascending and at least approximately perpendicular to the axis of the tubular element,

the feeding conduit is added to the tubular element in a removable manner,

the feeding conduit is further mounted to the tubular element by means of a clamp located on both sides of the edge of the proximal end of the tubular element,

the end at the distal side of the added feeding conduit has the shape of a taper,

the end at the distal side of the added feeding conduit is located in the vicinity of the deflection means,

the added feeding conduit comprises a plurality of independent distal feeding holes,

the added feeding conduit comprises a plurality of independent channels,
and/or for certain variants:

a feeding conduit for gas connects the auxiliary channel or channels of the device to the source or sources of respirable gases, said feeding conduit for gas comprising at the side of the source or sources a device for load loss arranged for limiting the flow rate and the pressure of said respirable gas which is available at the outlet of said source and for giving the jet of respirable gas a predetermined value of flow rate and a predetermined value of pressure, and comprising at the side of the auxiliary channel or channels a calibrated outlet valve arranged for connecting said feeding conduit for gas to the atmosphere as soon as the pressure in said feeding conduit for gas exceeds said predetermined value of the pressure,

the device for load loss is adjustable in order to allow for giving the jets of respirable gas a plurality of predetermined or pre-adjusted values of flow rate and pressure,

the calibration of the outlet valve is adjustable,

the device for load loss is incorporated in the tubular element,

the device for load loss is external to the tubular element,

the device comprises a humidifier located in the feeding conduit for gas connecting the source or sources of respirable gases to the auxiliary channel or channels,

the humidifier is located between the device for load loss and the calibrated outlet valve,
and/or for certain variants:

the device comprises a controlled valve which is likely to close the proximal end of the main channel at least partially and at least during insufflation of the respirable gas,

the controlled valve forms a monolithic assembly together with the tubular element,

the controlled valve is added to the tubular element,

the controlled valve is fixed on a flange capable to be fitted on the proximal end of the tubular element,

the controlled valve comprises a tight body having a toroidal section and located in the vicinity of the proximal end of the main channel and comprising at least one supple and resilient internal wall which, when it expands or when it retracts subsequent to the introduction or the evacuation of an inflating fluid into the tight body, controls the passage way of the main channel,

the free edge of the proximal end of the tubular element comprises at least one notch,

the device comprises at least one pressure tap located at the side of the distal end of the tubular element,

the pressure tap comprises an annular peripheral chamber which is coaxial to the tubular element and ends-out at the distal side of the device through an annular distal passage way, said annular peripheral chamber communicating with a lateral outlet nipple,

a fibrous or porous annular filter is located in the annular peripheral chamber,

the pressure tap is formed by the outlet hole through which one of the auxiliary channels, arranged in the wall of the tubular element, ends in the vicinity of the distal end of the tubular element,

the device comprises means receiving the pressure taken through said pressure tap and which is likely to command the opening of the controlled valve for opening the main channel,

the device comprises a calibrated outlet valve located at the proximal end of the tubular element, at the side opposite the free edge of the proximal end with respect to the controlled valve,

the introduction and evacuation of the inflating fluid into or out of the tight body results from the bi-directional movement of a volume of an appropriate fluid comprised in a buffer space of controllable variable volume,

the inflating fluid is a gas,
and/or for certain variants:

the device is specifically arranged for being implemented on a patient suffering from cardiac and respiratory arrest and on whom a massage of the thoracic cage is performed with phases of compression of the thoracic cage for exhaling and release phases of the thoracic cage (=decompression of the thoracic cage) for inhaling, and the device further comprises in its main channel at least one means for proximally slowing down the flow of air and/or oxygen inhaled through the proximal end at the beginning of inhaling in order to increase the depression inside the thoracic cage at the beginning of inhaling, said slowing down means being either a passive means or an active means, the passive means being chosen from one or more valves having a flexible membrane and/or one or more valves with flaps, the active means comprising an equipment for measuring the distal pressure, a control equipment, and a controlled equipment for reducing the passage way of a proximal part of the main channel, the control equipment being arranged for controlling the reduction of the passage way by the controlled means only at the beginning of inhaling when detected by the measurements obtained from the equipment for measuring the distal pressure,

the means for proximally slowing down is integrated into the device according to the invention,

the means for proximally slowing down is added to the proximal end of the device according to the invention,

the passive means for proximally slowing down comprises two valves with flap and spring, said valves being mounted in parallel or in series,

the passive means for proximally slowing down comprises a valve with a supple membrane,

the active means for proximally slowing down comprises a controlled equipment for reducing a passage way by means of inflatable balloons,

the equipment for measuring distal pressure is replaced by an equipment for thoracic measuring of the compressions of release of the massage of the thoracic cage.

The invention also relates to a nasal apparatus for respiratory assistance for a patient, comprising a device according to the invention for each nostril, both devices being supplied in parallel with respirable gas or gases, each device comprising a tubular element forming a main channel intended for being connected by its distal end to the respiratory system of the patient by one of his nostrils, said main channel connecting the patient's respiratory system by its proximal end to the outside, said device further comprising at least one auxiliary channel allowing for injection of a jet or of jets of respirable gas for the ventilation of said patient through distal outlet holes of the auxiliary channel or channels, said outlet holes leading to/opening-out in said main channel in the vicinity of the distal end of the latter, deflecting means, i.e. deflectors, allowing to deflect the gas jets toward the interior of said main channel, and the apparatus comprises at least one outlet hole shaped as an annular or semi annular strip resulting from uniting together at least some of the distal outlet holes, the thickness of the outlet hole shaped as a strip being less than 150 micrometers.

In different implementations of the apparatus, the following means, which can be used individually or according to any technically possible combination, are used:

the device incorporated in the apparatus has one or more of the characteristic features described with respect to said device,

the apparatus comprises a monolithic piece incorporating two devices according to the invention,

in the monolithic piece, the two tubular elements are parallel to one another,

the apparatus further comprises means for feeding a liquid to be nebulized in the gas flow passing the apparatus toward the patient,

the apparatus comprises, with respect to each tubular element, two portions of different diameters arranged in series, the portion having the greater diameter being proximal, i.e. at the side of free air opposite the side of the patient, and being provided with a lateral tubular connector,

the portions of greater and of smaller diameter are separated by a conical intermediate portion,

the apparatus comprises, for each portion of greater diameter, a sleeve located on the portion of greater diameter and arranging there a space with an annular section, the lateral tubular connector ending in said space with an annular section, means for fixing said sleeve to the portion of greater diameter which closes said space with an annular section at the proximal side opposite to a conical intermediate portion connecting the proximal portion of greater diameter to the portion of smaller diameter,

the apparatus comprises for each conical intermediate portion a lateral conduit for feeding liquid which ends in the conical intermediate portion in the vicinity of the connector of the latter and of the portion of smaller diameter,

each lateral conduit for feeding protrudes to the interior of the conical intermediate portion and has a taper with an inclination opposite to that of the conical intermediate portion, at the place where the lateral conduit ends,

the apparatus is provided with a device for feeding respirable gas or gases associated to a source of such gas or gases and provided with a pressure detector capable of detecting the beginning of exhalations and the beginning of inhalations of the ventilated patient, said feeding device further comprising, simultaneously fed by the same source, calibration means furnishing permanently a flow of respirable gas or gases under a first pressure capable of maintaining the bronchioli of the patient open, avoiding a collapse, and a controllable valve calibrated for furnishing, when it is open, a flow of respirable gas or gases under a second pressure which is higher than the first pressure and able to efficiently assist the inhalation of said patient, said controllable valve being controlled by said pressure detector, for closing during expirations and for opening during inhalations,

the feeding device comprises a flow meter mounted between the calibration means and the controllable valve, on the one hand, and the auxiliary channels, on the other hand,

the feeding device comprises an alarm device controlled by the pressure detector,

a pressure tap for the detector ends in at least one of the downstream tubular elements at the distal side, i.e. at the side of the patient, of said deflection means and in the vicinity of the distal end of said tubular element, said pressure tap being connected to the detector by a connecting tube,

the pressure tap is a hole going through the wall of the tubular element and the connecting tube is external to the latter,

the connecting tube is located inside the tubular element and the pressure tap is formed by a lateral notch formed in the connecting tube and having a length corresponding to several times the diameter of said connecting tube,

the apparatus comprises mobile elements capable of restrict the passage section of the gaseous jets at the deflection means,

the mobile elements are constituted by the distal ends of sleeves arranged for being able to slide inside the main channels under hard friction,

the apparatus comprises on each of its two distal ends, a nostril sheath, which is supple, arranged from the external, and intended to get into contact with the internal wall of the corresponding nostril,

each nostril sheath comprises an opening in front of the distal opening of the tubular element,

each nostril sheath is made of a foam material with open cells and closes the distal opening of the distal of the tubular element, while ensuring at the same time that respirable gas or gases and possible nebulized liquids can pass.

The invention also relates to a mask for respiratory assistance provided with a device according to the invention. The device related to the mask has one or more of the characteristic features described with respect to said device.

### BRIEF DESCRIPTION OF THE DRAWINGS

The present invention will now be described with respect to an example of an embodiment, without being limited to that example, and with respect to the following figures:

FIG. 1 is a diagrammatic and partial view, as an enlarged axial section, of a first embodiment of the device according to the invention,

FIGS. 2 and 3 are transverse sections, respectively along the lines II-II and of FIG. 1,

FIGS. 4 and 5 show, diagrammatically as an enlarged axial section, two variants of the distal end of the device according to the invention,

FIG. 6 shows, as a diagrammatic and partial axial section, a variant of the device according to the invention with two inverted flows,

FIG. 7 shows, as an axial section, another variant of the device of the invention, where the tubular element is not more intended to be directly introduced into the respiratory tract, but constitutes the flange of a mask for respiratory assistance intended to be applied to the face (mouth and/or nose) of a patient,

FIG. 8 shows an application according to the invention as a nasal apparatus for respiratory assistance for a patient,

FIG. 9 shows, as an axial sectional view, a variant of the device according to the invention,

FIGS. 10, 11, and 12 are transverse sections of the device of FIG. 9, respectively along the lines IV-IV, and V-V, and

FIG. 13 is a diagrammatic view, partially as an axial section, of a respiratory mask provided with a variant of the device according to the invention.

### DETAILED DESCRIPTION OF THE INVENTION

The device according to the invention allows intubation without stopping the respiratory assistance, injection of medicine, anaesthetics or humidity by respiratory way during the respiratory assistance, dynamic measuring of pressures, increase of the exchanged volume since the pressure is self-limited and there is no risk of squeezing the capillaries of the lungs. It allows, for the same quantity of exchanged oxygen, to decrease the percentage of oxygen in the mixture, which diminishes at the same time the secondary effects of the assistance, and opens the possibility to use less expensive respiratory systems than the ones used before.

The device for respiratory assistance according to the invention allows using the minimum difference of pressure necessary while furnishing a maximum of respirable gas. Further, the physical configuration of the device according to the invention, especially the main channel, allows keeping an internal diameter which is sufficient for allowing for passing the instruments and medical probes which the surgeon wants to use.

Generally speaking, the device according to the invention thus is constituted of a tubular element, generally a tube, which forms a main channel and which is intended to be connected by its distal end to the respiratory tract of the patient, whereas the proximal end of the tubular element ends at the ambient air outside the patient, the respiratory system of the patient thus being connected to the outside by means of the main channel of the device. Auxiliary channels are formed inside the thickness of the tubular element and open-out in the main channel. The auxiliary channels are fed with respiratory gas or gases by means of a feeding conduit. The device comprises deflection means in order to make the jets of respiratory gas or gases, which are injected by said auxiliary channels toward the main channel, converge to one another inside the main channel. Preferably, the means for deflecting the jets of respirable gas or gases deflects the jets toward the central axis of the main channel.

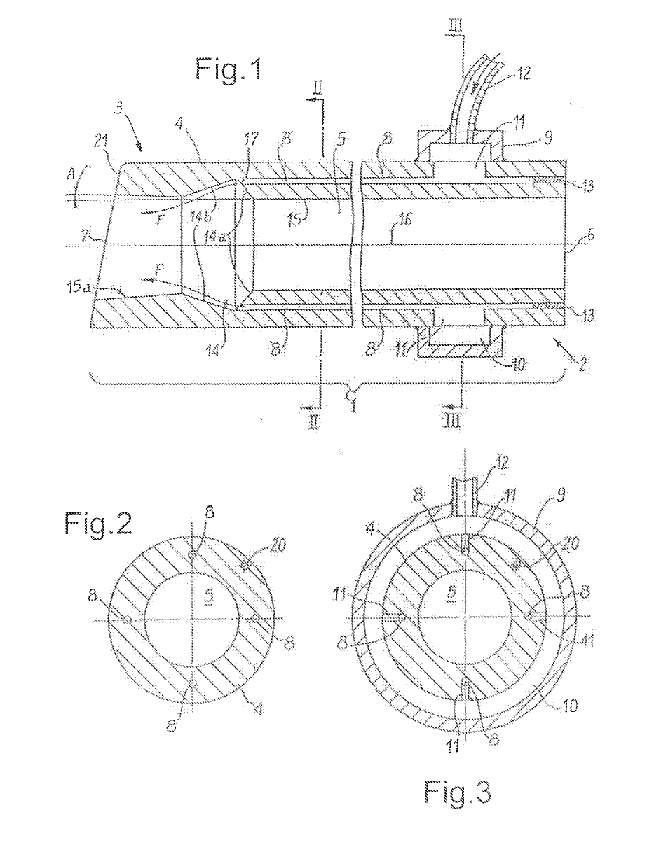

On FIG. 1, which is a diagrammatic view at a large scale, a tubular element is represented with its proximal end 2 and its distal end 3 of an embodiment 1 of the device according to the invention. That embodiment can constitute or be combined with, for example, an endotracheal probe, an oro-nasal probe with or without a balloon, an endotracheal paediatric probe, a monitoring probe for gases, an endobronchial probe, a nasopharyngeal probe, an anatomic intubation probe for children, a neonatal Cole probe, a cannula type Guedel probe, or a nasal probe for oxygen therapy.

The device 1 comprises a tubular element 4, which is supple or preformed, especially for adapting itself to the morphology of the patient, surrounding a main channel 5 which ends, by means of its proximal opening 6, at the proximal end 2 and, by means of its distal opening 7, at the distal end 3.

Thus, the main channel 5 is able to ensure a passage way between the openings 6 and 7, one of which, the distal opening 7, is intended to be inside the respiratory tract or ways of a patient or at the outside, then communicating with the latter ones, and the other one, the proximal opening 6, is intended to communicate with the outside environment of the patient. That proximal opening 6 can end at the ambient air and, in this case, the patient can inhale fresh air and exhale vicious air through the main channel 5. It is also possible to connect the proximal opening 6 at least to a source of pressurized respirable gas or gases (not shown) and provide a system of one-way valves, so that the patient inhales the respirable gas or gases of said source through the main channel 5 and exhales the vicious gas to the ambient air, through that main channel 5 as well.

The diameter of the main channel 5 is in the order of some millimeters. Satisfying trials have been performed with diameters of 3 mm, 7 mm, and 8 mm, and even a bit more.

Further, in the thickness of the wall of the tubular element 4, auxiliary channels 8 are arranged, which extend over nearly the whole length of the main channel 5. Those auxiliary channels 8 are intended to be connected to one or more sources of pressurized respirable gas (not shown). For example, that pressure is of some bars, e.g. 1, 2, or 4 bars, and it is adjustable. Preferably, as shown, those auxiliary channels are individualized channels over at least part of their way. According to variants, the auxiliary channels have a common portion forming a coaxial circular chamber outside the main channel or they are reduced, as far as their individual portion is concerned, to the outlet holes opening-out in the main channel. And, in this case, one can consider that there is one auxiliary channel, only. In any case, the auxiliary channel or channels open-out in the main channel by means of a number of outlet holes for forming there jets of respirable gas or gases.

As is shown on FIGS. 1 and 3, the connection to the source or sources of pressurized respirable gases can be implemented by means of a ring 9 surrounding in a tight manner the tubular element 4 at the side of the proximal end 2 and surrounding an annular tight chamber 10 around said tubular element. The auxiliary channels 8 are communicating with the chamber 10 thanks to local notches 11 of the wall of the tubular element 4 and said chamber 10 is connected to the source or sources of respirable gas or gases by means of a link 12. Of course, the proximal ends of the auxiliary channels 8 are closed, for example by means of stoppers 13.

The auxiliary channels 8 have a diameter which is smaller than the one of the main channel 5. The diameter of the auxiliary channels 8 is preferably close to or smaller than 150 microns in order to reduce or even suppress the noise generated by the gas jets in the main channel.

At the distal side, each one of the auxiliary channels 8 ends by means of a corresponding outlet hole 17 in a recess 14 of the internal wall 15 of the tubular element 4. The recess 14 is of annular shape and central in revolution to the central axis 16 of the tubular element at the distal end 3. It comprises a face 14a, which is approximately transverse or slightly tilted in order to constitute a widening of the main channel 5 into which said auxiliary channels 8 open-out by means of their outlet holes 17, as well as a face 14b following the face 14a and converging toward the axis 16.

Preferably, between the converging tilted face 14b and the distal opening 7, the internal wall 15 has a portion 15a which is slightly widened toward the outside, as is indicated by an angle A on FIG. 1.

Thus, when the auxiliary channels 8 are supplied with pressurized respirable gas via the elements 9 through 12, the corresponding gaseous jets hit the tilted face 14b which deflects them toward the axis 16 (arrows F on FIG. 1), thus forming deflection means, and generating in the vicinity thereof a depression zone favoring the gas circulation inside the main channel 5 from the proximal opening 6 to the distal opening 7. In that way, the patient's inhaling is promoted.

One understands that it is possible to form the auxiliary channels in the device in a way which is different from the one shown as an example. Thus, for example, the auxiliary channels can be notches 11 or grooves over the whole length rather than to follow as channels integrated in the wall of the tubular element as shown, said notches being externally closed by an added external tubular wall applied to the tubular element. Said external tubular wall can also form deflection means at the outlet holes of the auxiliary channels.

Preferably, the distance between each of the outlet holes 17 of the auxiliary channels and the distal opening 7 is in the order of 1 to 2 cm.

Downstream from the distal opening 7, the pressure in the pulmonary cage is low and practically constant.

Thus, thanks to the invention, one obtains a respiratory assistance which is non aggressive to the patient and with a nearly total disappearing of a dead space inherent to the known probes.

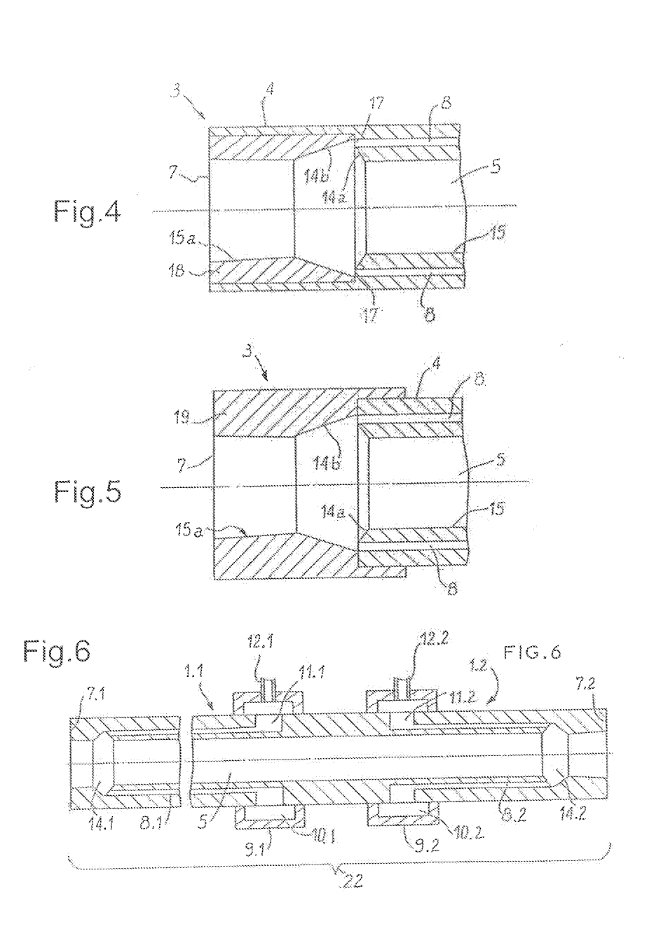

In the embodiment of the invention shown on FIG. 1, it has been indicated that faces 14a and 14b have been obtained by recesses made in the internal wall 15 of main channel 5. It is obvious that this embodiment is not limiting and that the faces 14a 14b can be obtained in different ways. On FIGS. 4 and 5, for example, face 14a is formed in the internal wall 15 of the tubular element 4, whereas face 14b is provided on a flange 18 or 19 which fits internally, flange 18, or externally, flange 19, on the tubular element 4.

Of course, in that case, the distal opening 7 and the diverging wall 15a are respectively held by flange 18 or 19.

As is shown on FIGS. 2 and 3, the auxiliary channels 8 are evenly located around the axis of the tubular element 4. Their number varies according to the use (adult or child), but it is generally comprised between ten and twenty. In a general manner, the number of outlet holes of the auxiliary channel or channels and the diameter of the outlet holes of the auxiliary channel or channels are adjusted so as to obtain a sufficient flow rate for the respirable gas or gases.

It will be noted that at least one of the auxiliary channels 8, instead of being connected commonly with the other ones to the source of pressurized respirable gas (by means of elements 9 through 12), can be fed continuously from the source of respirable gas, in order to maintain a positive pressure in the lungs of the patient, and this during or at the end of the exhaling phase triggered by the insufflation of gas into the auxiliary channels 8 (anti-collapse effect).

According to still another variant of the invention, one of the auxiliary channels 8 can also be specialized for providing a medical fluid or a humidifying fluid, if the pressurized source does not have the characteristics as required.

In order to obtain humidification, the auxiliary channel providing water, preferably lukewarm water, is preferably U-shaped at its distal end and ends in a recess arranged in the internal wall 15, in which recesses further ends a channel providing pressurized air. In that hole or recess which is preferably located between the recess 14 and the distal opening 7, the water channel and the air channel end in front of one another, that means practically on the same axis, both fluids, i.e. air and water, arriving in opposite directions, which allows for vaporizing the water, and the vaporized water then being taken away by the introduced air.

At least one additional channel 20 can be provided in the thickness of the tubular element 4 which ends in the distal end face 21 of the tubular element 4 and serves for lodging a device for measuring pressure (not shown).

When there are at least two pressure taps, especially one at each end of the tubular element, they allow to calculate the flow rate of the gas, based on the difference between the measured pressures.

The tubular element 4 can comprise, at the distal end 3, an inflatable balloon (not shown) which is provided with the necessary security devices or any other balloon allowing to work as a security valve in case of overpressure in the lungs. That possible balloon can be inflated by a supplemental channel (not shown) which is associated to the tubular element 4.

A security device can be simply constituted by an elastic sleeve surrounding the tubular element and partially glued to the latter, and covering a hole formed through the wall of said tubular element, especially in the vicinity of the proximal end. Thus, when the internal pressure is too high, the gas can evacuate through said hole and then between the external wall of the tubular element and the internal wall of the elastic sleeve. When a security sleeve is also provided in the vicinity of the distal end, the corresponding hole, located beyond a possible balloon for maintaining by friction, has to put into communication the interior of the tubular element with the ambient air; said balloon then has to be by-passed what can be obtained, for example, by placing it around the elastic sleeve.

The variant 22 of the embodiment of the device according to the invention, which is with two inverted flows, shown on FIG. 6, comprises two devices 1.1 and 1.2, each having a structure similar to the one of FIG. 1, which are inverted and put together at their proximal ends, the device 1.2 may then be shorter than the device 1.1. In that variant of embodiment 22, the distal hole (at the side of the patient) of the device is constituted by the distal opening 7.1 of device 1.1, whereas the proximal hole (opposite the patient) is formed by the opening 7.2 of device 1.2. Each one of the devices 1.1 and 1.2 is provided with its supply system 9.1, 9.2, 10.1, 10.2, 11.1, 11.2, and 12.1, 12.2 for pressurized respirable gas, supplying the respective channels 8.1 or 8.2, leading to/opening-out in the annular holes 14.1 or 14.2, respectively close to said openings 7.1 and 7.2. The device 22 forms a two-reverse-flow probe. That device being placed on the patient, i.e. the device 1.1 being connected to the respiratory tract of the latter, the device 1.2 and the rings 12.1 and 12.2 being outside the patient, alternatingly channels 8.1 are fed through the elements 9.1 through 12.1 and the channels 8.2 are fed through the elements 9.2 through 12.2 with pressurized respirable gas or gases, in order to promote alternatingly inhaling and exhaling of the patient.

To this end, a (not shown) device for communication and for adjusting the flow rate and the insufflation time of gas is connected to the rings 9.1 and 9.2, on the one hand, and to the gas source, on the other hand.

The tubular respiratory element, as constituted that way, allows for respiratory assistance of inhaling and a respiratory assistance for exhaling, provided that a continuous flow be maintained in order to provide for security and limiting the risk of pulmonary collapse.

The pressure in the channels 8.2 is advantageously higher than the pressure in the channels 8.1, since the effect of taking along the fluid which is present in the probe, is not of the same nature.

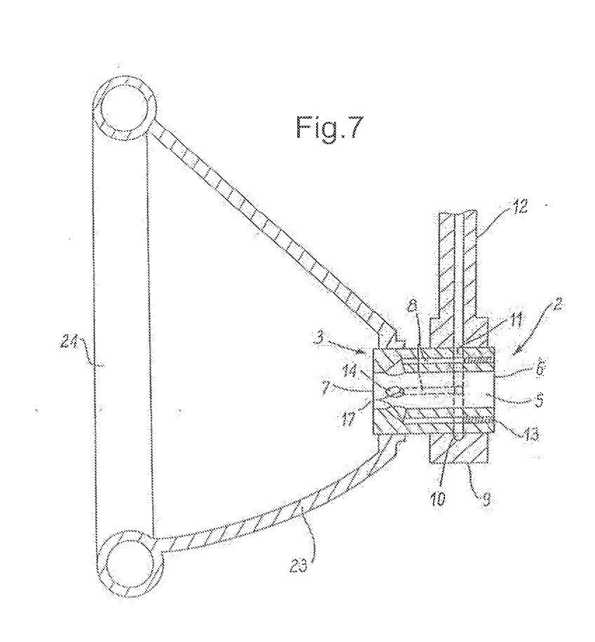

The device of FIG. 7 differs from the device of FIGS. 1 through 6 on the one hand by the fact that the tubular element 1 is not intended to be introduced into or connected to a respiratory tract, but constitutes the flange of a mask, and on the other hand by the fact that the dimensions, especially length and diameters, are different. According to FIG. 7, the main tubular element constitutes the flange for air getting in and out of a mask which, besides, is of a commonly known type, i.e. mainly comprising a shell 23, a bead 24 for tightness, and (not represented) fixing means such as belts.

FIG. 7 also shows a variant of the embodiment of hole 14, where the deflection means does not form a continuous annular recess, but is constituted by a discontinuous number of generally conical holes formed in the internal wall 15, and at the bottom of each one of them leads to/opens-out, by means of its outlet hole, the distal end of an auxiliary channel 8.

The principle of how the device of FIG. 7 works is the same as the one described further up with respect to FIGS. 1 through 6.

Further, although FIG. 7 comprises a tubular element where the respiratory assistance only works for the direction of inhaling (as is the case of the device of FIG. 1), a mask can also be provided with a two-inverse-flows device according to FIG. 6, for assistance for both inhaling and exhaling.

The tubular element 4, on which the embodiments of the device according to the invention are based, can be made of all material already used for respiratory probes, for example of a polyvinyl chloride, possibly with a coating made of silicone or of steel allowing for high-pressure injections.

Of course, the dimensions of the device according to the invention may vary a lot, especially as a function of how the tubular element is applied, and of the size of the patient who might be an adult, a child, a baby or a premature.

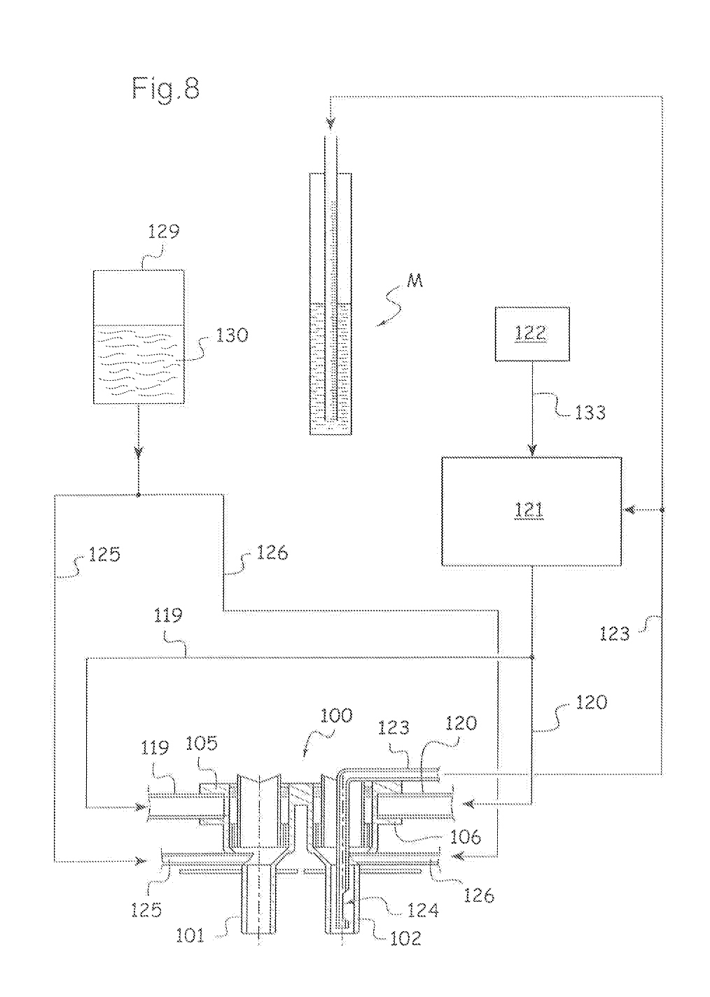

The nasal apparatus or appliance for respiratory assistance of FIG. 8 uses one device according to the invention per nostril, both appliances then being arranged in parallel. FIG. 8 shows that diagrammatically with an apparatus mounted in its circuit for providing respirable gas or gases and a humidifying liquid. The apparatus comprises two devices according to the invention inside a monolithic tubular piece 100.

Lateral tubular connections 105 and 106 are respectively connected to feeding conduits 119 and 120, which are supplied in parallel with a respirable ventilation gas by a feeding device 121 which is itself connected to a source 122 of such a gas by a conduit 133.

Further, a capillary tube 123 is introduced into in the right main channel of FIG. 8, the distal end of that tube being provided with a notch 124 having a length which is several times the diameter of the capillary tube 123. That lateral notch 124 of the capillary tube is located in the distal part of the main channel and serves as a pressure tap at that place. The capillary tube 123 transmits the pressure to the feeding device 11 and to a pressure meter with water M.

Each tubular element is further associated to a feeding conduit shaped as a lateral conduit 125 or 126 ending in a conical intermediate portion of the corresponding main channel between a portion of larger diameter and a distal portion of smaller diameter of the main channel.

The ends of the lateral conduits 125 and 126 may protrude to the interior of the main channel and have a taper as shown. The inclination of the taper is opposite to the one of the conical intermediate portion.

The lateral conduits 125 and 126 are connected in parallel to a tank 129 containing a liquid 130, for example water, maybe with additional medicine or an analogous product, and which are supplied with a liquid from that tank, for example by gravity or by capillarity.

From what has been described up to now, it is easy to conclude that, when a patient starts inhaling, after having exhaled, the feeding device 121 receives the corresponding pressure difference by the capillary tube 123 and is able to send to the patient continuous or pulsed jets of respirable gas or gases by means of the feeding conduits 119 and 120. The jets of respirable gas or gases, which are formed inside each main channel, are deflected by the conical intermediate portions, taking along with them droplets of the liquid 130 sticking to the tapers of the ends of the lateral conduits 125 and 126 and get humidified.

Contrary to this, when a patient starts exhaling, after having inhaled, the feeding apparatus is stopped by the pressure transmitted by the capillary tube 123 and the patient can exhale freely through the main channels.

Disks made of foam (not shown) may be fitted around the distal ends 101, 102 of the tubular elements and serve then as a soft stop for the introduction of a monolithic piece 100 into the nostrils of the patient.

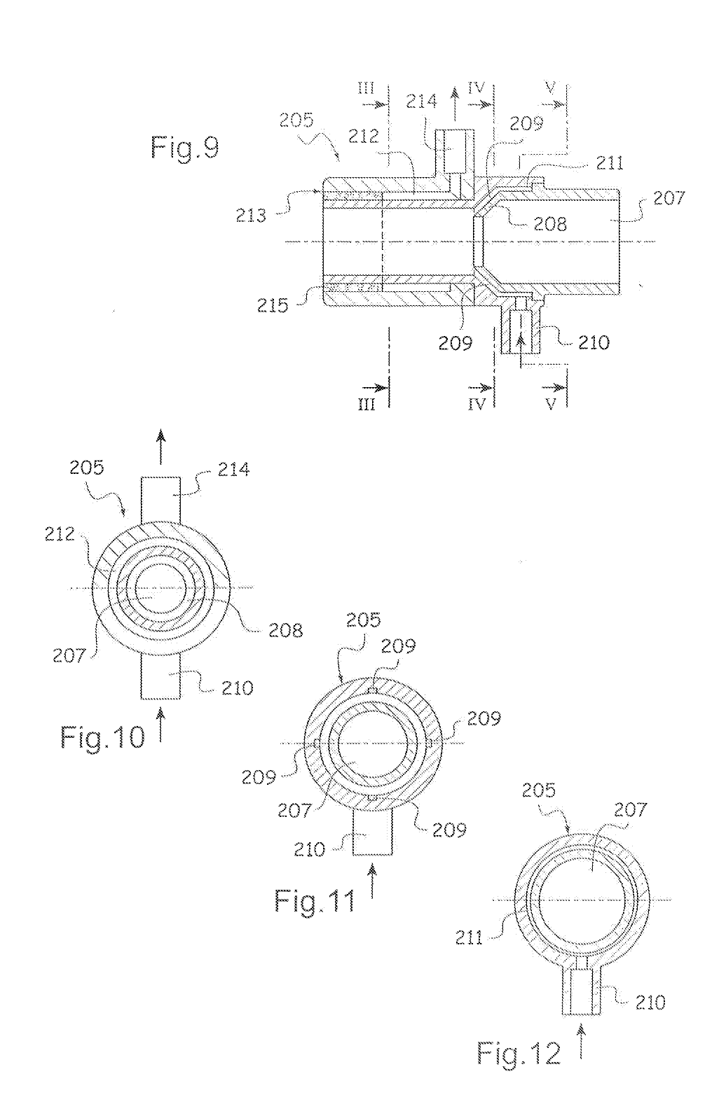

The variant 205 of the device, shown in detail on FIGS. 9 through 12, comprise an internal main channel 207 and, in a median portion, a conical wall 208, protruding into the interior of said main channel 207. The conical wall 208 which is a deflector is intended for deflecting the jets of respirable gas or gases toward the axis of the main channel 207, said jets being injected by the auxiliary channels 209, fed from a feeding nipple 210 by means of a peripheral annular chamber 211.

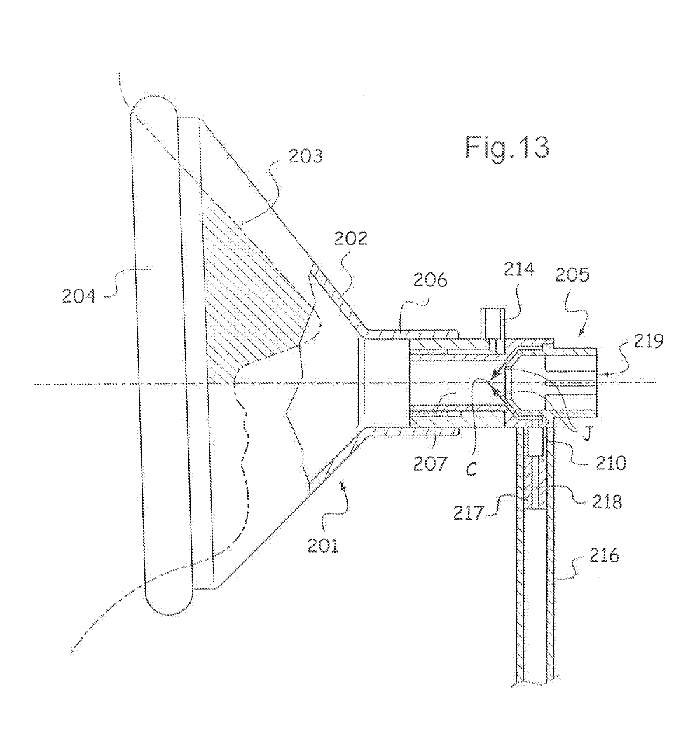

Further, at the distal side, the device 205 comprises a peripheral annular chamber 212 coaxial to the device 205. The peripheral annular chamber 212 ends at the distal side by means of a distal annular passage way 213. When the device 205 is installed on a facial mask (shown on FIG. 13), the peripheral annular chamber 212 communicates with the interior of the mask through that distal annular passage way 213. The peripheral annular chamber 212 communicates at the proximal side with an outlet nipple 214.

A fibrous or porous filter 215, for example made of cotton, synthetic foam or other, is located in the peripheral annular chamber 212 for absorbing the gaseous turbulences and, consequently, the too big variations of pressure. Indeed, the outlet nipple 214 can be connected, for example, to a gas analyzer and/or a device for measuring pressure. Of course, the connections between the outlet nipple 214 and the gas analyzer and/or the device for measuring pressure are designed in order for the device to not influence the analysis and/or the measurement of pressure via the connection, by the consumption of the gas to be analyzed.

Thus, the practitioner assisting the patient knows permanently the composition of the gas inside the mask, especially its content of carbonic gas, and the pressure inside said mask. He can thus take the appropriate measures for intervention depending on said composition and pressure of the gas.

One understands that the variant 205 can be made in different ways, thereby obtaining the same functions of respiratory assistance and of measurements. Further, the device can also comprise additionally means or conduits for injection of medicine and/or water, especially for nebulization.

More generally, the device according to the invention can be applied in many other ways, for example combined with a nasal probe, a buccal probe, a tracheal probe, etc.

The facial respiratory mask 213, shown on FIG. 13, comprises a rigid shell 202 of generally frustoconical shape, intended for being applied to the face 203 of a patient by means of a cushion 204 covering its peripheral opening. At the side opposite the face, the mask 201 is provided with a device for respiratory assistance according to the present invention, comprising a tubular element 205, fixed to said shell 202 or fitted on a tubular protrusion 206 of the latter. The tubular element 205 serves as an inlet and outlet flange for gas in that mask 201, its proximal end, opposite the patient, being at the ambient air, whereas its distal end, at the side of the patient, is connected to the mask 201. As before, the tubular element 205 forms an internal main channel 207 and comprises, at a median portion, deflection means, i.e. deflectors, oriented toward the central axis of the main channel. The deflection means is intended for deflecting jets J of respiratory gas toward the central axis of said main channel, the jets being injected by outlet holes at the ends of the peripheral auxiliary channels, supplied from a feeding nipple 210, by means of a peripheral annular chamber, said jets of respiratory gas converging toward a converging point C of the central axis of said main channel 207. The respiratory gas is conducted to the peripheral annular chamber and to the peripheral auxiliary channels by a feeding conduit 216 which is connected at one side to the feeding nipple 210 and at the other side to source of respiratory gas or gases such as a bottle of gas under pressure (not shown). Inside the feeding conduit 216 is arranged a stopper 217 which is punched with a longitudinal passage way 218. For example, the stopper 217 can be obtained by cutting lengthwise a portion of a profile having a longitudinal passage way with a constant diameter d. It can easily be understood that by varying the length of the stopper and its portion, the load loss applied to the feeding conduit 216 by said stopper is varied, too. Thus, it is possible to adjust said load loss by trial such that the pressure of the respiratory gas at the distal end of the tubular element 205, in use, corresponds to an optimal oxygenation, free of danger, of the patient. Further, the tubular element 205 comprises a nipple 214 for taking gas and/or for measuring pressure. In that embodiment, obstacles are provided in the proximal end of the tubular element 205, for example converging fins 219, avoiding accidental introduction of an object that, otherwise, could hermetically close said proximal end.

As already seen, the device according to the invention can be used combined with a facial mask. However, the device of the invention can more generally be used combined with all other type of apparatus used for respiratory assistance, such as, for example, a laryngeal mask, and also in any position, especially supraglottic or supratracheal.

The device of the invention can be used for attempts of reanimation of persons suffering from respiratory and/or cardiac arrest. Those persons can produce liquid spilling such as expectoration, bleeding, hemoptysis, even vomiting if they are not yet intubated, etc., said spillings being liable to pass into the respiratory ways and to be pushed out through the device, including the risk of spreading them through the proximal output/end and to create possibly contaminating soiling. Therefore, in a variant, means for filtering/retaining such liquid spilling at the proximal end of the device are provided. That means can be a filter, for example an adaptor in which a filtering mass is or can be installed, said mass can be, for example, a pad having a texture which allows gases to pass. That means can be a separator with a recipient for separation, preferably at a distance of the device and connected to the latter by a flexible tube. If the device further comprises additional functional means at the proximal side, for example slowing down means, then, preferably, the means for filtering/retaining is located between the device and those additional functional means. In a simpler variant, a tube is simply connected to a proximal end of the device by a first end and the other end of the tube is positioned at a place where possible soiling is not of any importance and/or can be removed.

Finally, in a variant of embodiment disclosed in the International Application WO2008113913, which can be used for the present invention, the device further comprises slowing down means for slowing down spontaneously or in a controlled manner the inlet of ambient air into the main channel via its proximal end. This means can be installed permanently on the device and is then an integral part of said device, or, otherwise, be removable by connecting it to the proximal end of the device. Thanks to this slowing down means, it is possible to use the device for reanimation of a person suffering from cardiac arrest. In this case, the device with means for slowing down is used for ventilation, for example, by means of a mask or by intubation, and compressions and decompressions are alternatingly performed on the thoracic cage of said person. Then, the intake of ambient air into the device and thus toward the patient is slowed down at the beginning of each decompression. Consequently, the pressure in the thoracic cage and on the heart decreases and, as a result, there is assistance for cardiac expansion and for flowing back of blood to the heart. The spontaneous proximal lowering is essentially passive and implements calibrated valves which open and close according to a pressure difference. The controlled proximal lowering is essentially active and a controlled means for restricting the main channel is implemented. That restriction means is controlled according to measures obtained from a sensor detecting at least a beginning of decompression/release of a massage of the thoracic cage. For variants, the passive or active proximal slowing down can be combined with a modulation of the jets of the auxiliary channels based on inhaling and exhaling cycles, and the proximal slowing down can even be replaced by a modulation of the jets, since an increase of the speed of the jets corresponds to increasing the distal valve effect and create or increase the distal slowing down of the flow of gases of the main channel, and vice versa when the jets are reduced. It can be understood that the distal slowing down by the jets of the auxiliary channels is active and, in practice, the pressure and the flow rate will be increased in the auxiliary channels at a beginning of inhaling detected by a sensor.

1-15. (canceled)

16. Device for respiratory assistance of a patient comprising a tubular element forming a main channel intended for being connected by its distal end to a respiratory tract of the patient, said main channel connecting the patient's respiratory system by its proximal end to the outside, said device further comprising at least one auxiliary channel allowing for injection of a jet or of jets of respirable gas through one or more distal outlet holes of the auxiliary channel or channels, said jet or jets of respirable gas being intended for ventilation of the patient, said one or more outlet hole opening-out in the main channel in the vicinity of the distal end of the latter, deflectors allowing to deflect the jets of respirable gas toward the interior of said main channel,
wherein it comprises art least one outlet hole shaped as an annular or semi annular strip resulting from uniting together at least some of the distal outlet holes, the thickness of the outlet hole shaped as a strip being less than 150 micrometers.

17. Device according to claim 16, wherein the thickness of each outlet hole shaped as an annular or semi annular strip is between 50 micrometers and 10 micrometers.

18. Device according to claim 16, wherein the thickness of each outlet hole shaped as an annular or semi annular strip is about 25 micrometers.

19. Device according to claim 16, wherein it comprises, in addition to the outlet holes shaped as annular or semi annular strips, individual outlet holes, said individual holes having a diameter of less than 150 micrometers.

20. Device according to claim 16, wherein the outlet holes shaped as a strip or as strips result from uniting together at least certain ones of the distal outlet holes and of their corresponding auxiliary channels.

21. Device according to claim 16, wherein it comprises a group of auxiliary channels, said auxiliary channels being approximately parallel to the central axis of said tubular element on at least part of their way.

22. Device according to claim 19, wherein it comprises a single common auxiliary channel to which the outlet hole or holes are connected, said common auxiliary channel being an external annular passage way approximately coaxial and parallel to the central axis of said tubular element.

23. Device according to claim 16, wherein it comprises at least one crown of outlet holes, the outlet holes of one crown being located along a transverse section of said tubular element.

24. Device according to claim 16, wherein the deflection means is arranged as to allow the jets to be oriented toward the distal end of the tubular element with an inclination with respect to the central axis of said tubular element which is between 90°, the axis of the jets then being perpendicular to said central axis, and 25°, the axis of the jets then crossing the central axis with an angle of 25°.

25. Device according to claim 16, wherein it is arranged as a two-inverted-flows device, allowing for favoring both exhaling and inhaling of a patient, the device comprising at least one supplemental auxiliary channel, which is independent from the first auxiliary channel or channels of the jets of the distal end of the main channel and connected to a source of pressurized gas, said at least one supplemental channel opening-out in the main channel in the vicinity of the proximal end of the latter, and at least the proximal end or ends of said supplemental auxiliary channel or channels which open-out with corresponding proximal outlet holes in the main channel, is or are parallel to it, whereas in front of each proximal outlet hole of the corresponding supplemental auxiliary channel, deflectors are provided for deflecting the gas jet passing through the latter toward the interior of said main channel.

26. Device according to claim 16, wherein it further comprises at least one feeding tube, supplied with a liquid substance for being nebulized into the main channel, and a nebulization means.

27. Device according to claim 16, wherein it further comprises at least one pressure tap located at the side of the distal end of the tubular element.

28. Device according to claim 16, wherein it further comprises at least one pressure tap located at the side of the distal end of the tubular element and that the pressure tap comprises an annular peripheral chamber which is coaxial to the tubular element and ends at the distal side of the device by an annular distal passage way, said annular peripheral chamber being connected to a lateral outlet nipple, an annular fibrous or porous filter being located in the annular peripheral chamber.

29. Device according to claim 16, wherein it is specifically arranged for being used for a patient suffering from cardiac and respiratory arrest and to whom a massage of the thoracic cage is applied with phases of compression of the thoracic cage for exhaling and release phases for inhaling, and in that it further comprises in its main channel at least one means for proximally slowing down the flow of air and/or oxygen inhaled through the proximal end at the beginning of inhaling in order to increase the depression inside the thoracic cage at the beginning of inhaling, said slowing down means being either a passive means or an active means, the passive means being chosen from one or more valves having a flexible membrane and/or one or more valves having a flap, the active means comprising an equipment for measuring the distal pressure, a control equipment, and a controlled equipment for reducing the passage way of a proximal part of the main channel, the control equipment being arranged for controlling the reduction of the passage way by the controlled means only at the beginning of inhaling detected by the measurements obtained from the equipment for measuring the distal pressure.

30. Apparatus for respiratory assistance to be fixed on the nose of a patient, wherein it comprises for each nostril a device according to any of the preceding claims, both devices being supplied in parallel with respirable gas or gases, each device comprising a tubular element forming a main channel intended for being connected by its distal end to the respiratory system of the patient by one of his nostrils, said main channel connecting the patient's respiratory system by its proximal end to the outside, said device further comprising at least one auxiliary channel allowing for injection of a jet or of jets of respirable gas for the ventilation of said patient through distal outlet holes of the auxiliary channel or channels, said outlet holes opening-out in said main channel in the vicinity of the distal end of the latter, deflectors allowing to deflect the gas jets toward the interior of said main channel, and in that it comprises at least one outlet hole shaped as an annular or semi annular strip resulting from uniting together at least some of the distal outlet holes, the thickness of the outlet hole shaped as a strip being less than 150 micrometers.

31. Mask for respiratory assistance, wherein it comprises a device according to claim 16.
